(19) United States
(12) Patent Application Publication
(10) Pub. No.: US 2010/0063477 A1
(43) Pub. Date: Mar. 11, 2010

(54) Medical Tubing with a Flexible Tip and Method of Preparation

(75) Inventor: Atsushi Ohigawa, Fukuroi-shi (JP)

Correspondence Address: TYCO HEALTHCARE - EDWARD S. JARMOLOWICZ, 15 HAMPSHIRE STREET, MANSFIELD, MA, 02048, US

(73) Assignee: Tyco Healthcare Group LP, Mansfield, MA (US)

(21) Appl. No.: 12/550,849

(22) Filed: Aug. 31, 2009

(30) Foreign Application Priority Data
Sep. 11, 2008 (JP) 2008-232875

(51) Int. Cl. A61M 25/00; B32B 37/06

(52) U.S. Cl. 604/523; 156/60

(57) ABSTRACT

Medical tubing having a flexible tip comprising medical tubing formed from a polyurethane resin, and a flexible tip formed from a mixture comprising a styrene-ethylene-butylene-styrene block copolymer, polypropylene, polyurethane, and mineral oil. The flexible tip may be formed from a mixture that comprises 20-40% by weight of the styrene-ethylene-butylene-styrene block copolymer, a total of 15-50% by weight of polypropylene and polyurethane, and 30-45% by weight of mineral oil. Also, a method for producing medical tubing having a flexible tip comprising medical tubing formed from a polyurethane resin, and a flexible tip formed from a mixture comprising a styrene-ethylene-butylene-styrene block copolymer, polypropylene, polyurethane, and mineral oil, comprising fusion bonding the medical tubing and the flexible tip.

### TECHNICAL FIELD OF THE DISCLOSURE

The present disclosure relates to medical tubing with a flexible tip that is suitable for use for various purposes, such as the excretion of waste liquids from the body of a patient, or for the supply of liquid drugs, blood or other liquids to the body of a patient. The present disclosure also relates to a method for producing the medical tubing with the flexible tip.

### BACKGROUND OF THE DISCLOSURE

Conventionally, materials typically used to form medical tubing include polyurethane resin, silicone resin and vinyl chloride resin. Medical tubing made from polyurethane tubing has a certain degree of hardness, and when pushed or twisted these forces are readily transmitted to the tip so it is easily manipulated and also, it softens at body temperature and otherwise has superior biocompatibility, and also it is resistant to splitting. Medical tubing made from silicone resin is flexible but may be too soft and may lack ease of manipulation. Medical tubing made from vinyl chloride resin is inexpensive. Thus, the type of tubing is selected depending on the circumstances based on the characteristics of each.

Such medical tubing is required to be less irritating when inserted into the body, have good ease of insertion that permits it to be bent and easily inserted into convoluted portions of the body, and be able to return to its original shape after being bent. For these reasons, medical tubing with a flexible tip where the tubing body is given a certain amount of hardness and the tip is given flexibility is used. This medical tubing with a flexible tip such as a catheter for intra-intestinal insertion has a constitution wherein a guide made of flexible tubing is provided at the tip of the tubing body.

However, with the aforementioned medical tubing with a flexible tip, the tubing body is formed from soft polyvinyl chloride resin, urethane resin, polyamide resin, polyester resin or other resin materials, while the guide is made from silicone rubber, urethane resin, natural rubber latex resin or other flexible resin. For this reason, if the tubing body is made of polyurethane and the guide is made of silicone rubber, while this is preferable from the standpoint of hardness, it is difficult to bond the two by welding or adhesive. In addition, if urethane-based resins are used for both, while fusion-bonding becomes possible, there is no longer much of a difference in hardness. With the other combinations also, it is not possible to obtain medical tubing with a flexible tip that is easily bonded and that is provided with a preferable difference in hardness. For this reason, there are problems in that the manufacturing process for the aforementioned medical tubing with a flexible tip may become complex or the manufacturing costs may become high, the durability may become poor or the outside diameter of the adhered portion may become large.

In consideration of these circumstances, the present disclosure has as an object to provide medical tubing with a flexible tip that has superior ease of insertion and that is easily manufactured.

### SUMMARY OF THE DISCLOSURE

A medical tubing having a flexible tip is provided comprising medical tubing formed from a polyurethane resin, and a flexible tip formed from a mixture comprising a styrene-ethylene-butylene-styrene block copolymer. Also provided is a method for producing medical tubing having a flexible tip wherein the medical tubing is formed from a polyurethane resin, and the flexible tip is formed from a mixture comprising a styrene-ethylene-butylene-styrene block copolymer, polypropylene, polyurethane, and mineral oil, comprising fusion bonding the medical tubing and the flexible tip.

### BRIEF DESCRIPTION OF THE DRAWINGS

FIG. 1 is a side view of the medical tubing with a flexible tip according to one embodiment of the present invention.

FIG. 2 is a partial-cutaway side view illustrating the state of bonding of the tubing body forming intermediate and tip forming intermediate in hot-air welding.

FIG. 3 is a side view illustrating the ends of the tubing body forming intermediate and tip forming intermediate.

Corresponding reference numerals indicate corresponding parts throughout the drawings, and herein the following reference numerals apply:

10 . . . Medical tubing with a flexible tip

11 . . . Tubing body

12 . . . Tip

### DETAILED DESCRIPTION OF THE DISCLOSURE

A medical tubing having a flexible tip is provided comprising medical tubing formed from a polyurethane resin, and a flexible tip formed from a mixture comprising a styrene-ethylene-butylene-styrene block copolymer. Also provided is a method for producing medical tubing having a flexible tip wherein the medical tubing is formed from a polyurethane resin, and the flexible tip is formed from a mixture comprising a styrene-ethylene-butylene-styrene block copolymer, polypropylene, polyurethane, and mineral oil, comprising fusion bonding the medical tubing and the flexible tip.

In order to achieve the aforementioned object, the medical tubing with a flexible tip according to the present disclosure is made by the steps of forming a tubing body using a tubing body forming material, forming a tip using a tip forming material that is softer as measured by Shore Hardness, than said tubing body forming material, and unitizing by, preferably, fusion-bonding said tubing body and said tip thus formed. The medical tubing with a flexible tip is characterized in that said tubing body forming material comprises a polyurethane resin, and said tip forming material comprises a forming material made by mixing a styrene-ethylene-butylene-styrene block copolymer, polypropylene, polyurethane and mineral oil. Any method for fusion bonding may be utilized. For example, the fusion bonding may be performed by hot-air welding, heating using a radiofrequency heater or laser heater, or the like.

Any polyurethane resin, preferably one that is typically used to make medical tubing, is used as the tubing body forming material. Moreover, the tip is made from a forming material made by mixing a styrene-ethylene-butylene-styrene block copolymer, polypropylene, polyurethane and mineral oil, hereinafter referred to as a “SEBS blend material,” that has a lower Shore Hardness value (is softer) than the Shore Hardness value of the polyurethane resin. The polyurethane resin is easily manipulated, softens at body temperature and otherwise has superior biocompatibility and also is resistant to splitting, among other advantages. For this reason, a polyurethane resin is the material used to constitute the tubing body that makes up the greater part of the medical tubing with a flexible tip. The SEBS blend material that constitutes the flexible tip of the medical tubing, provides flexibility and is easily formed, and moreover it has good chemical resistance, among other advantages.

Moreover, the polyurethane resin and the SEBS blend material have good mutual solubility at a temperature of about 200° C. Accordingly, by making the tubing body from the polyurethane resin and the tip from the SEBS blend material, one can obtain medical tubing with a flexible tip that has superior ease of insertion and good durability because of its high bonding strength, and that is easily manufactured. In addition, a good bond is possible even if the outside diameter of the bonded portion is small. For this reason, when the medical tubing with a flexible tip is inserted into a narrow, bending portion of the body such as the intestinal tract, the tip is able to bend so as to follow along the bending portions of the intestinal tract, and smooth insertion becomes possible. In addition, the tip is provided with flexibility so it will not irritate or injure the intestinal tract. Moreover, after the tip has passed through a bent portion, it quickly reverts to its original shape.

In another embodiment of the disclosure of the medical tubing with a flexible tip, the tip forming material made by mixing a styrene-ethylene-butylene-styrene block copolymer, polypropylene, polyurethane and mineral oil comprises 20-40% by weight of the mixture, of styrene-ethylene-butylene-styrene block copolymer, 30-45% by weight of the mixture, of mineral oil, and a total of 15-50% by weight of the mixture, of polypropylene and polyurethane. By setting the proportion of mineral oil to 30-45% by weight of the mixture, it is possible to obtain a flexible tip of the medical tubing that has superior flexibility.

In another embodiment of the medical tubing with a flexible tip according to the present disclosure, the proportion by weight of each of the polypropylene and the polyurethane included in said tip forming material is 1% or more. The proportion of polypropylene and polyurethane can be varied appropriately depending on use.

In another embodiment of the medical tubing with a flexible tip according to the present disclosure, the mineral oil is a paraffinic oil, a naphthenic oil, a higher fatty acid or a mixture thereof. Thereby, the mineral oil can exhibit a superior effect as a softening agent

### DETAILED DESCRIPTION OF THE DRAWINGS

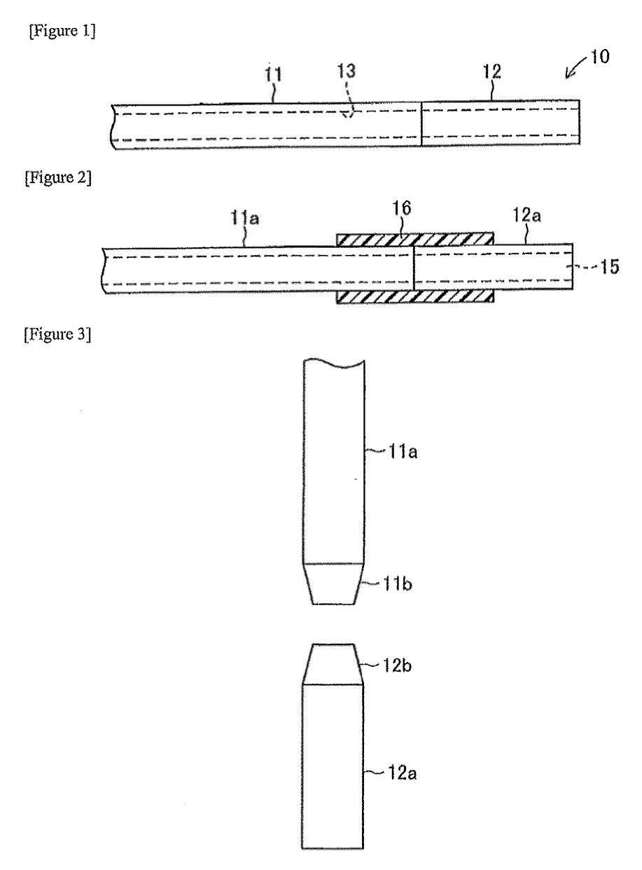

The following is a description of an embodiment of the present invention made with reference to the drawings. FIG. 1 shows medical tubing with a flexible tip 10 according to this embodiment. The medical tubing with a flexible tip 10 comprises a tubing body 11 made from a polyurethane resin and a tip 12 made from a SEBS blend material made by mixing a styrene-ethylene-butylene-styrene block copolymer, polypropylene, polyurethane and mineral oil. This medical tubing with a flexible tip 10 may be used for many purposes, such as the excretion of waste liquids from the body of a patient, or for the supply of liquid drugs, blood or other liquids to the body of a patient, and so a lumen 13 constituting a liquid channel is formed in the interior.

The SEBS blend material constituting the tip 12 may comprise 20-40% by weight of the mixture of styrene-ethylene-butylene-styrene block copolymer, 30-45% by weight of the mixture of mineral oil, and a total of 15-50% by weight of the mixture of polypropylene and polyurethane, and the proportion by weight of each of the polypropylene and polyurethane included in said tip forming material is 1% or more. The tip 12 of the medical tubing with a flexible tip 10 used in this specific embodiment comprises 30% by weight of the mixture of styrene-ethylene-butylene-styrene block copolymer, 38% by weight of the mixture of mineral oil, and the proportion by weight of polypropylene and polyurethane is 16% each, with a total of 32%.

A paraffinic oil is used as the mineral oil of the SEBS blend material.

The medical tubing with a flexible tip 10 comprises tubing with an outside diameter of 2-8 mm and an inside diameter of 0.5-5 mm, and the length of the tubing body 11 is set to 500-4000 mm and the length of the tip 12 is set to 3-50 mm. In addition, the Shore Hardness of the polyurethane resin constituting the tubing body 11 was 70 A to 70 D, while the Shore Hardness of the SEBS blend material constituting the tip 12 was 30 A to 70 A. The tip 12 is made of material that has a lower Shore Hardness value and is softer than that of the tubing body 11.

The medical tubing with a flexible tip 10 is prepared by forming each of a soft thermosetting resin material made of polyurethane and the SEBS blend material in an extrusion molding machine (not shown) into tube shapes. The end surfaces of both tubular bodies are then fusion bonded by means of hot-air welding. In this instance, as shown in FIG. 2, around the outside surface of a pin 15 made of a stainless-steel rod, are placed a pre-bonding tubing body forming intermediate 11a, which is to constitute the tubing body 11, and a pre-bonding tip forming intermediate 12a, which is to constitute the tip 12, and the end surfaces of both are placed in contact with each other.

The diameter of pin 15 is the same or slightly smaller than the inside diameter of the tubing body forming intermediate 11a and tip forming intermediate 12a. As shown in FIG. 3, both the tip portion 11b of the tubing body forming intermediate 11a and the tip portion 12b of the tip forming intermediate 12a are thinner at the tip. This tip portion 11b and tip portion 12b are placed in contact with each other. In FIG. 2, the tip portion 11b and tip portion 12b are not shown.

Next, a cylindrical heat-shrinking tube 16 that is able to adequately cover from the outside the contacting portions of the tubing body forming intermediate 11a and the tip forming intermediate 12a as well as portions in the vicinity is used to cover contacting portions of the tubing body forming intermediate 11a and the tip forming intermediate 12a as well as portions in the vicinity. This heat-shrinking tube 16 may be made of silicone resin or polyethylene terephthalate, for example, and has the property of shrinking to a fixed size when heat is applied. Then, a hot-air heater (not shown) is used to blow hot air at a temperature of approximately 200° C. at the contacting portions of the tubing body forming intermediate 11a and the tip forming intermediate 12a as well as portions in the vicinity that are covered by the heat-shrinking tube 16.

Thereby, heat-shrinking tube 16 shrinks and its inside diameter becomes about the same as the outside diameter of the medical tubing with a flexible tip 10 shown in FIG. 1. Then, both the tip portion 11b of the tubing body forming intermediate 11a and the tip portion 12b of the tip forming intermediate 12a fuse and are bonded together, By removing the heat-shrinking tube 16, medical tubing with a flexible tip 10 with the tubing body forming intermediate 11a and tip forming intermediate 12a unitized is obtained. In this instance, the heat-shrinking tube 16 shrinks such that its inside diameter becomes about the same as the outside diameter of the medical tubing with a flexible tip 10. Thus, even at the time of fusion, the tubing body forming intermediate 11a and the tip forming intermediate 12a can maintain about the same outside diameter. The method of fusion-bonding is not limited to hot-air welding. The fusion-bonding can also be performed by any known technique, such as by heating using a radiofrequency heater or laser heater. Preferable methods may be used as appropriate depending on the shape and size and other aspects of the medical tubing with a flexible tip 10.

In this manner, with the medical tubing with a flexible tip 10 according to this embodiment, the tubing body 11 is made of polyurethane resin having the property of superior ease of manipulation, while the tip 12 is made of the SEBS blend material having flexibility. For this reason, when the medical tubing with a flexible tip 10 is inserted into certain portions within the body, the tip 12 is able to bend so as to follow the portion into which it is inserted and thus smooth insertion becomes possible. In addition, the polyurethane resin and SEBS blend material have good mutual solubility at a temperature of about 200° C., so the tubing body forming intermediate 11a made of polyurethane resin and the tip forming intermediate 12a made of SEBS blend material bond readily with each other and thus it is possible to obtain a durable medical tubing with a flexible tip 10 having high bonding strength.

The present disclosure is not limited to the aforementioned embodiment but rather may include appropriate modifications. For example, the tip 12 of the medical tubing with a flexible tip 10 in the aforementioned embodiment comprises 30% by weight of the mixture of styrene-ethyleiie-butylene-styrene block copolymer, 38% by weight of the mixture of mineral oil, and the proportion by weight of polypropylene and polyurethane is 16% each. The proportions by weight may be appropriately changed such as, for example, within the ranges of 20-40% by weight of the mixture of styrene-ethylene-butylene-styrene block copolymer, 30-45% by weight of mineral oil, and a total of 15-50% by weight of the mixture of polypropylene and polyurethane.

While the mineral oil used in the aforementioned embodiment is a paraffinic oil, it is possible to alternatively use a naphthenic oil or a higher fatty acid.

The medical tubing with a flexible tip according to the present disclosure may be used as digestive tract tubing, enteral nutrition tubing, intestinal depressurization tubing, drainage tubing or the like.

What is claimed is:

1. Medical tubing having a flexible tip comprising medical tubing formed from a polyurethane resin, and a flexible tip formed from a mixture comprising a styrene-ethylene-butylene-styrene block copolymer, polypropylene, polyurethane, and mineral oil.

2. The medical tubing having a flexible tip according to claim 1, wherein the flexible tip is formed from a mixture that comprises 20-40% by weight of the styreiie-ethylene-butylene-styrene block copolymer, a total of 15-50% by weight of polypropylene and polyurethane, and 30-45% by weight of mineral oil.

3. The medical tubing with a flexible tip according to claim 2, wherein the proportion by weight of each of the polypropylene and the polyethylene is at least 1%.

4. The medical tubing with a flexible tip according to claim 1, wherein the mineral oil is a paraffinic oil, a naphthenic oil, a higher fatty acid, or a mixture thereof.

5. The medical tubing with a flexible tip according to claim 2, wherein the mineral oil is a paraffinic oil, a naphthenic oil, a higher fatty acid, or a mixture thereof.

6. The medical tubing with a flexible tip according to claim 3, wherein the mineral oil is a paraffinic oil, a naphthenic oil, a higher fatty acid, or a mixture thereof.

7. A method for producing medical tubing having a flexible tip comprising medical tubing formed from a polyurethane resin, and a flexible tip formed from a mixture comprising a styrene-ethylene-butylene-styrene block copolymer, polypropylene, polyurethane, and mineral oil, comprising fusion bonding the medical tubing and the flexible tip.

8. The method according to claim 7, wherein the flexible tip is formed from a mixture that comprises 20-40% by weight of the styrene-ethylene-butylene-styrene block copolymer, a total of 15-50% by weight of polypropylene and polyurethane, and 30-45% by weight of mineral oil.

9. The method according to claim 8, wherein the proportion by weight of each of the polypropylene and the polyethylene is at least 1%.

10. The method according to claim 7, wherein the mineral oil is a paraffinic oil, a naphthenic oil, a higher fatty acid, or a mixture thereof.

11. The method according to claim 8, wherein the mineral oil is a paraffinic oil, a naphthenic oil, a higher fatty acid, or a mixture thereof.

12. The method according to claim 9, wherein the mineral oil is a paraffinic oil, a naphthenic oil, a higher fatty acid, or a mixture thereof.

13. The method according to claim 7, wherein the fusion bonding is performed by hot air welding, a radiofrequency heater, or a laser heater.

14. The method according to claim 13, wherein the fusion bonding is performed by hot air welding.
